(19) United States
(12) Patent Application Publication
(10) Pub. No.: US 2021/0093163 A1
(43) Pub. Date: Apr. 1, 2021

(54) ENDOSCOPE WITH INTEGRATED TISSUE ACQUISITION CAPABILITY

(71) Applicant: Boston Scientific Scimed, Inc., Maple Grove, MN (US)

(72) Inventors: James J. Scutti, Norwell, MA (US); John T. Favreau, Spencer, MA (US); Ethan Miller, Ashland, MA (US); Peter L. Dayton, Brookline, MA (US)

(21) Appl. No.: 17/029,401

(22) Filed: Sep. 23, 2020

Related U.S. Application Data
(60) Provisional application No. 62/906,310, filed on Sep. 26, 2019.

(51) Int. Cl. A61B 1/00; A61B 17/04; A61B 1/018; A61B 1/015

(52) U.S. Cl. CPC A61B 1/00087; A61B 17/0469; A61B 2017/06076; A61B 1/015; A61B 1/00066; A61B 1/018

(57) ABSTRACT

A tissue closure system includes an endoscope having first and second lumens therethrough, first and second control elements disposed within the first and second lumens, respectively, and a tissue engaging portion integral with a distal end of the endoscope. The tissue engaging portion includes a tissue acquisition assembly and a tissue closure assembly. The tissue acquisition assembly is controllable via the first control element, and the tissue closure assembly controllable via the second control element. The tissue engaging portion includes a tissue grasping portion for engaging first and second target tissue portions disposed adjacent an opening in target tissue. The tissue closure assembly is configured to pierce the target tissue, and to hold the first and second target tissue portions together to close the opening in the target tissue.

### CROSS-REFERENCE TO RELATED APPLICATIONS

The present application claims the benefit of priority under 35 U.S.C. § 119 to U.S. Provisional Patent Application 62/906,310, filed Sep. 26, 2019, which application is incorporated herein by reference in its entirety for all purposes.

### FIELD

The present disclosure pertains to medical devices. More particularly, the present disclosure pertains to devices, systems, and methods for closing tissue, such as a tissue defect or wound resulting from an endoscopic procedure, e.g., a mucosal resection or endoscopic submucosal dissection procedure.

### BACKGROUND

A tissue or opening or defect, for example a wound, such as a tissue defect within a body lumen resulting from endoscopic mucosal resection (EMR) or endoscopic submucosal dissection (ESD), may be large enough that it may be difficult to close on its own, or the time for it to close may be prolonged. A risk of infection may increase the longer the wound remains open.

It is with the above considerations in mind that the improvements of the present disclosure may be useful.

### SUMMARY

The present disclosure, in its various aspects, relates to endoscopic devices and methods for acquiring and manipulating target tissue, and for closing opening in the target tissue.

A tissue closure system is disclosed, including an endoscope having first and second lumens therethrough, first and second control elements disposed within the first and second lumens, respectively, and a tissue engaging portion integral with a distal end of the endoscope. The tissue engaging portion may include a tissue acquisition assembly and a tissue closure assembly. The tissue acquisition assembly may be controllable via the first control element, while the tissue closure assembly may be controllable via the second control element.

The tissue engaging portion may include a tissue grasping portion for engaging first and second target tissue portions disposed adjacent an opening in target tissue. The tissue closure assembly may be configured to pierce the target tissue and to hold the first and second target tissue portions together to close the opening in the target tissue.

The tissue grasping portion may include first and second pluralities of prongs oriented such that distal portions of each of the first and second pluralities of prongs are engageable with the first and second target tissue portions. The first and second pluralities of prongs can be offset relative to each other along a length of the tissue grasping portion so that when the tissue grasping portion is moved from a first configuration to a second configuration, the distal portions of the first and second pluralities of prongs are operable to press sections of tissue into spaces between opposing adjacent prongs, causing the first and second target tissue portions to form a wavy overlapping seam therebetween.

The tissue grasping portion can include first and second rollers. The first roller may be engageable with the first target tissue portion, while the second roller may be engageable with the second target tissue portion. The first and second rollers can be rotatable in opposite directions to cause the first and second target tissue portions to be drawn between the first and second rollers to press the first and second target tissue portions together. The first and second rollers can include a roughened, spiked or adhesive surface.

The tissue acquisition assembly can include a chamber, and the first control element can include a suction channel to apply suction to the chamber. When the chamber overlies the opening in the target tissue, the suction may draw the first and second target tissue portions into the chamber, pressing the first and second target tissue portions together. The tissue closure assembly may include a needle having a corkscrew shape so that as the needle is passed along a seam between the first and second target tissue portions the needle and a fastener pass alternately through the first and second target tissue portions to close the opening.

The tissue grasping portion may include a distal tip portion having a suction channel disposed therethrough. The suction channel may be in fluid communication with a suction source. The distal tip portion may be made from a flexible material for pressing against the target tissue. The distal tip portion can be engageable with the target tissue such that the suction channel overlies the tissue opening to engage the first and second target tissue portions. Suction applied via the suction channel may cause the first and second target tissue portions to be drawn into the suction channel to press the first and second target tissue portions together. The first and second target tissue portions drawn into the suction channel cause the distal tip portion to transition from a generally cylindrical shape to a shape having an expanded outer diameter “EOD” that is greater than an outer diameter “OD” of the distal tip portion prior to suction being applied via the suction channel.

The tissue grasping portion may further include a sleeve that is movable over the distal tip portion to compress the distal tip portion, thereby causing first and second fasteners disposed in the distal tip portion to pierce the first and second target tissue portions and to lock to each other, closing the opening. The first and second fasteners may be first and second staple assemblies. The tissue closure system may also include opposing first and second hammer assemblies positioned on opposite sides of the suction channel so that when the sleeve is moved over the distal tip portion to compress the distal tip portion, the first and second hammer assemblies engage the first and second staple assemblies to pierce the first and second target tissue portions and to lock to each other. In some embodiments, the tissue engaging portion includes a central portion housing a plurality of said first and second staple assemblies.

The tissue grasping portion may include first and second arms having first and second distal portions engageable with the first and second target tissue portions. The first and second arms each may have a first curvature so that the first and second arms are disposable on opposite sides of a longitudinal axis of the first lumen. The first and second arms each may have a second curvature so that the first and second arms are disposed away from the longitudinal axis and toward the target tissue.

In some embodiments the first and second arms may be made from a super-elastic material. The tissue grasping portion may be configurable in a first elongated configuration when the tissue grasping portion is disposed in the first lumen. When the tissue grasping portion is moved out of the first lumen the first and second arms may automatically reassume the first and second curvatures.

An endoscope is disclosed, including a handle having first and second actuators, a flexible tube having an articulable distal end, and a tissue engaging portion integral to the distal end of the flexible tube, the tissue engaging portion comprising a tissue acquisition assembly and a tissue closure assembly. The tissue engaging portion may include a tissue grasping portion for engaging first and second target tissue portions disposed adjacent an opening in target tissue. The tissue closure assembly may be configured to pierce the target tissue, and to hold the first and second target tissue portions together to close the opening in the target tissue.

The tissue grasping portion may include first and second pluralities of prongs oriented such that distal portions of each of the first and second pluralities of prongs can engage the first and second target tissue portions. The first and second pluralities of prongs may be offset relative to each other along a length of the tissue grasping portion so that when the tissue grasping portion is moved from a first configuration to a second configuration, the distal portions of the first and second pluralities of prongs are operable to press sections of tissue into spaces between opposing adjacent prongs, causing the first and second target tissue portions to form a wavy overlapping seam therebetween.

The tissue grasping portion may include first and second rollers. The first roller may be engageable with the first target tissue portion, while the second roller may be engageable with the second target tissue portion. The first and second rollers may be rotatable in opposite directions to cause the first and second target tissue portions to be drawn between the first and second rollers to press the first and second target tissue portions together.

The tissue closure assembly may include a needle comprises a corkscrew shape so that as the needle is passed along a seam between the first and second target tissue portions the needle and a fastener pass alternately through the first and second target tissue portions to close the opening.

The tissue grasping portion can include a distal tip portion having a suction channel disposed therethrough. The suction channel may be in fluid communication with a suction source. The distal tip portion may be made from a flexible material for pressing against the target tissue. The distal tip portion may be engageable with the target tissue such that the suction channel overlies the tissue opening to engage the first and second target tissue portions. Suction applied via the suction channel may cause the first and second target tissue portions to be drawn into the suction channel to press the first and second target tissue portions together.

The first and second target tissue portions drawn into the suction channel may cause the distal tip portion to transition from a generally cylindrical shape to a shape having an expanded outer diameter “EOD” that is greater than an outer diameter “OD” of the distal tip portion prior to application of a the suction. The tissue grasping portion may further include a sleeve that is movable over the distal tip portion to compress the distal tip portion, thereby causing first and second fasteners disposed in the distal tip portion to pierce the first and second target tissue portions and to lock to each other, closing the opening.

The tissue grasping portion may include first and second arms having first and second distal portions engageable with the first and second target tissue portions. The first and second arms each has a first curvature so that the first and second arms are disposable on opposite sides of a longitudinal axis of a first lumen of an endoscope. The first and second arms may have a second curvature so that the first and second arms are disposable away from the longitudinal axis and toward the target tissue.

The tissue acquisition assembly may include a chamber and a first control element, where the first control element comprises a suction channel to apply suction to the chamber. When the chamber overlies the opening in the target tissue, the suction may be configured to draw the first and second target tissue portions into the chamber, pressing the first and second target tissue portions together.

A method is disclosed for closing an opening in tissue. The method may include positioning a distal end of an endoscope adjacent to an opening in tissue, the distal end including an integral tissue acquisition assembly, engaging first and second target tissue portions on opposing sides of a tissue opening using a grasping portion of a tissue acquisition assembly, where the grasping portion associated with the distal end of the endoscope, holding the first and second target tissue portions together using the grasping portion, piercing the first and second target tissue portions using a tissue closure assembly, and engaging the first and second target tissue portions using a fastener to close the opening in tissue.

In some embodiments, engaging first and second target tissue portions includes engaging the first and second target tissue portions using first and second pluralities of prongs, where the first and second pluralities of prongs being offset relative to each other along a length of the tissue grasping portion, and moving the tissue grasping portion from a first configuration to a second configuration so that distal portions of the first and second pluralities of prongs press sections of tissue into spaces between opposing adjacent prongs causing the first and second target tissue portions to form a wavy overlapping seam therebetween.

In some embodiments, engaging first and second target tissue portions includes applying suction to the first and second target tissue portions to draw the first and second target tissue portions into a position accessible by a tissue closure assembly of the endoscope to engage the first and second target tissue portions using the fastener.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

Non-limiting examples of the present disclosure are described by way of example with reference to the accompanying figures, which are schematic and not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of skill in the art to understand the disclosure. In the figures:

FIG. 1A is an illustration of an example endoscope according to an embodiment of the present disclosure.

FIG. 1B is a perspective view of an embodiment of a device in accordance with the present disclosure.

FIGS. 2A-2F are a series of side and top plan views of an embodiment of a device in accordance with the present disclosure.

FIGS. 3A-3F are a series of side and end views of an embodiment of a device in accordance with the present disclosure.

FIGS. 4A-4E are a series of top views of an embodiment of a device in accordance with the present disclosure.

FIGS. 5A-5D are a series of side views of an embodiment of a device in accordance with the present disclosure.

FIGS. 5E-5G are perspective, top plan, and side views, respectively, of a gripping portion of the embodiment of FIG. 5A.

FIGS. 6A and 6B are side and end views, respectively, of an embodiment of a device in accordance with the present disclosure.

FIGS. 6C-6E are perspective views of the embodiment of FIGS. 6A and 6B engaging example tissue.

It is noted that the drawings are intended to depict only typical or exemplary embodiments of the disclosure. Accordingly, the drawings should not be considered as limiting the scope of the disclosure.

### DETAILED DESCRIPTION

Various embodiments according to the present disclosure are described below. As used herein, “proximal end” refers to the end of a device that lies closest to the medical professional along the device when introducing the device into a patient, and “distal end” refers to the end of a device or object that lies furthest from the medical professional along the device during implantation, positioning, or delivery.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used in connection with other embodiments whether or not explicitly described unless clearly stated to the contrary.

The detailed description should be read with reference to the drawings, which are not necessarily to scale, depict illustrative embodiments and are not intended to limit the scope of the invention.

The disclosure is not limited to the particular embodiments described, as such may vary. The terminology used herein is for the purpose of describing particular embodiments only and is not intended to be limiting beyond the scope of the appended claims. Unless defined otherwise, all technical terms used herein have the same meaning as commonly understood by one of ordinary skill in the art to which the disclosure belongs. Finally, although embodiments of the present disclosure are described with specific reference to medical devices and systems and procedures relating to tissues of the digestive system, it should be appreciated that such medical devices and methods may be used to dissect tissues of the abdominal cavity, gastrointestinal system, thoracic cavity, urinary and reproductive tract and the like. Moreover, a variety of medical procedures may benefit from the presently disclosed medical devices and procedures, including, for example, Endoscopic Submucosal Dissection (ESD), Peroral Endoscopic Myotomy (POEM), cholecystectomy and Video-Assisted Thorascopic Surgery (VATS) procedures. The structures and configurations, and methods of deploying, in order to stabilize, manipulate and provide a clear field of view may find utility beyond dissection.

As used herein, the term “distal” refers to the end farthest away from a medical professional when introducing a device into a patient, while the term “proximal” refers to the end closest to the medical professional when introducing a device into a patient.

As used herein, the term “target tissue” refers to tissue remaining after removal of an unhealthy, diseased (i.e., cancerous, pre-cancerous etc.) or otherwise undesirable portion of tissue that may be healthy or unhealthy. The removed tissue may also include tissues that are suspected of being unhealthy or diseased, but which require surgical removal for verification of their disease status by biopsy. “Target tissue” thus includes tissue on opposing sides of an opening resulting from removal of portions of tissue.

The disclosure pertains to medical devices, e.g., endoscopes, gastroscopes, bronchoscopes, colonoscopes, ureteroscopes, and the like, having integrated features for acquiring, manipulating, and closing openings in target tissue. Although single-use endoscopes are described herein, it is understood that embodiments of the present disclosure may be included in reusable medical devices such as endoscopes as well. In some instances, the devices disclosed for acquiring, manipulating, and closing openings in target tissue may be configured such that they may be used within a single working or available channel of the endoscope, and in some embodiments may be operated by a single individual, although in some embodiments a second individual may be involved. In some embodiments, the devices described herein may be considered as operating along a single line of operation. The devices themselves may be translatable distally and proximally within a working channel, and a handle portion may have one or more controls adapted to manipulate the devices in a desired manner. The device may be configured to enable selective acquisition, manipulation, and closure of openings in the target tissue.

The trend in medicine is moving from laparoscopic and open surgical procedures to miniaturized, endoscopic procedures. The Endoscopist can perform ever more complex non or minimally invasive procedures under direct visualization. Current endoscopes provide working channels to enable the use of dedicated instruments for such treatments, but they may not include the intrinsic capability to treat and manipulate tissue being accessed and examined. There exists a need for endoscopes, colonoscopes, duodenoscopes, bronchoscopes, gastroscopes, uteroscopes, or similar medical devices which possess specific built-in treatment capabilities. Such devices can facilitate both a broad range of procedural interventions that are becoming more prevalent in hospitals and can lead to the development of significantly more capable and complex scope designs.

Further, infection prevention controls in the clinical setting create a demand for single-use scopes which mitigate the risk of patient infection and associated adverse events. For example, currently commercial duodenoscopes often include distal tips having complex mechanical features. Such complexity can make it difficult to properly disinfect reusable scopes between procedures.

The disclosed systems, methods and devices include an endoscope in which complex functionality is integrated into the design of the endoscope. Such complex functionality includes one or more of tissue grasping and suturing, stapling, clip deployment, and the like. The disclosed systems, methods and devices can be of a single-use design that can aid in infection prevention. That is, the disclosed endoscopes, which provide solutions to complex noninvasive clinical needs, can minimize or eliminate infection risks because they can be sterile, single-use devices.

The disclosed devices include single disposable devices that integrates the visualization and navigation capabilities of current endoscopes with the ability to acquire target tissue and close the tissue using either sutures or staples, or other mechanical fasteners. It will be appreciated that the disclosed devices may be used to close openings in tissue having a wide range of sizes, and thus are not limited in that regard.

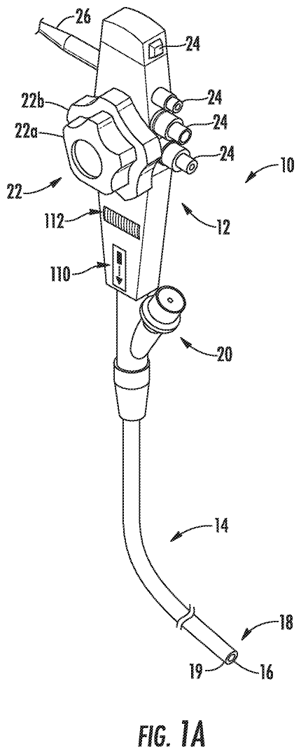

An example endoscope 10 is illustrated in FIG. 1A. The endoscope 10 may be any of a number of types of endoscopes or related medical devices usually identified by the particular anatomy desired to be reached. For example, the endoscope 10 may be a colonoscopes, duodenoscopes, bronchoscopes, gastroscopes, uteroscopes, or similar medical devices. The endoscope 10 may include a handle portion 12 and an elongate shaft 14 extending distally from the handle portion 12 to a distal tip 18. The shaft 14 may include a lumen defining a working channel 16 extending through the shaft 14 from a distal end 19 near the distal tip 18 of shaft 14 to an access port 20 that may be positioned in the handpiece 12 or another portion of the endoscope 10. Although the endoscope 10 is depicted with a single working channel in FIG. 1A, it can be appreciated that in other embodiments, the endoscope 10 may include multiple working channels, as desired.

In some cases, the handle portion 12 may include one or a plurality of controls 22, such as rotating knobs, which may be used to control one or more aspects of the distal tip 18 of the shaft 14 during operation. For example, a first rotating knob 22a may control up and down movement or deflection of the distal tip 18 of the shaft 14, while a second rotating knob 22b may control side-to-side movement or deflection of the distal tip 18 of the shaft 14. The handle portion 12 may also include one or a plurality of buttons 24, which may be used to activate suction or deliver fluid such as air, saline and/or water, etc. through a lumen of the endoscope 10 or perform other functions as desired. Additionally, in some cases, the handle portion 12 may include an optical cable 26 connected to an external light source (not shown). An imaging element, such as a charge coupled device (CCD) camera can be disposed at the distal tip to enable a user to visualize a working area.

FIG. 1B is a perspective view of a distal tip of a device 100 according to the present disclosure that may be used for acquiring (i.e., grasping or gripping) target tissue and may also be used to close openings in the target tissue. As mentioned, the device 100 may be an endoscope, bronchoscope, gastroscope, ureteroscope, colonoscope, or the like, having at least one lumen, and a working channel for providing suction, insufflation, additional tool access, and the like. The device 100 may have a handle portion 12 and an elongate shaft 14 (FIG. 1A) coupled to a tissue engaging portion 104 disposed at, and integral with, the distal tip 18. It will be appreciated that there is a change in scale on either side of the break line shown. In some embodiments, the tissue engaging portion 104 may comprise a tissue acquisition assembly 106 and a tissue closure assembly 108. The handle portion 12 may extend proximally from the tissue engaging portion 104. In some embodiments, the handle portion 12 may include a first actuator such as a thumb slide 110 that can be manipulated by a user. The handle portion 12 may also include a second actuator such as a thumb wheel 112. In some embodiments, as will be described, the first and second actuators 110, 112 may be used to actuate the tissue acquisition assembly 106 and the tissue closure assembly 108. Communication between the first and second actuators 110, 112 and the tissue acquisition and closure assemblies 106, 108 may be via associated lumens 114, 116.

In use, one of the first and second actuators 110, 112 may be used to actuate the tissue acquisition assembly 106, which may include at least one element configured to grasp opposing tissue portions of a tissue opening and to hold the opposing tissue portions together. The other of the first and second actuators 110, 112 may then be used to actuate the tissue closure assembly 108 to apply a suture, clip, or the like to the opposing tissue portions.

The device 100 for use with any/all of the embodiments disclosed herein may include a variety of additional functionalities, including steerability (e.g., up to four directions with 180-degree rotatable tip), air insufflation, water/fluid flush, a fiberoptic light source, and/or video visualization via an imaging chip disposed at or near the tissue engaging portion 104.

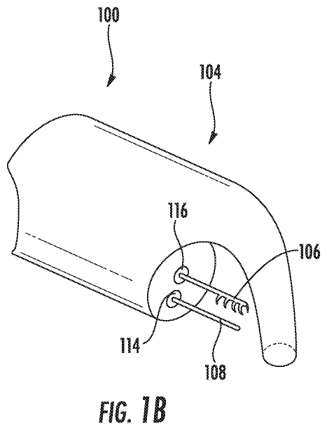

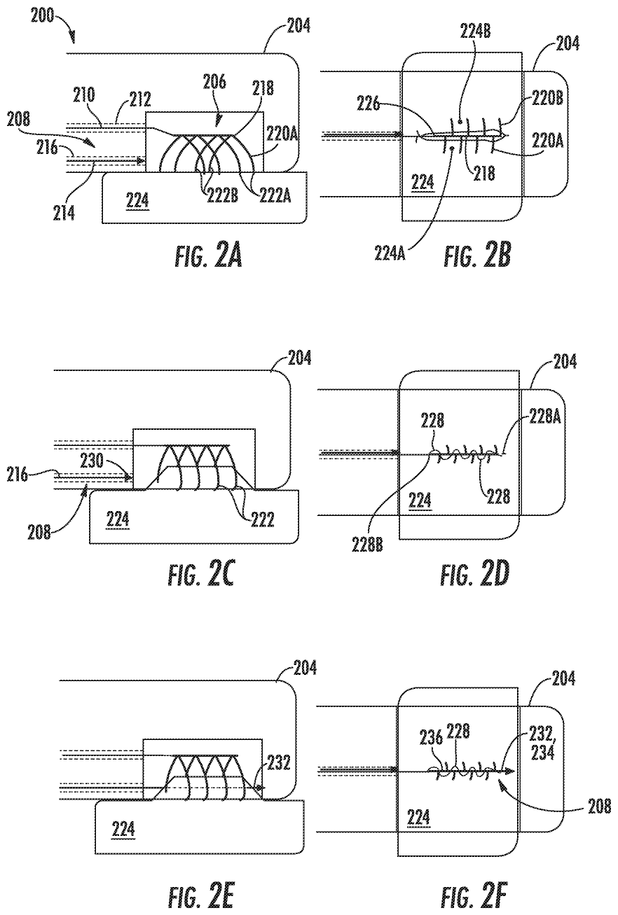

FIGS. 2A-2F illustrate an example tissue engaging portion 204 according to the present disclosure. As will be appreciated the tissue engaging portion 204 of this embodiment may be part of an endoscopic system that includes some or all the features and functionalities described in relation to the device 100 of FIG. 1. Moreover, the functionality of grasping target tissue and closing an opening in the target tissue may be conducted by a user manipulating one or both of the first and second actuators 110, 112 and/or by actuating other control mechanisms not illustrated.

The tissue engaging portion 204 may include a tissue acquisition assembly 206 and a tissue closure assembly 208. The tissue acquisition assembly 206 may be controlled via a first control element 210 disposed within a first lumen 212 in the endoscope 200, while the tissue closure assembly 208 may be controlled via a second control element 214 disposed within a second lumen 216 in the endoscope 200. The first and second control elements 210, 214 may be coupled to the first and second actuators 110, 112 (FIG. 1A) to enable a user to manipulate the tissue acquisition assembly 206 and the tissue closure assembly 208 via the user handle portion 12. The first and second control elements 210, 214 may comprise wires, cables, or similar structures having axial and rotational stiffness to enable the user to manipulate the tissue acquisition assembly 206 and the tissue closure assembly 208.

In the illustrated embodiment, the tissue acquisition assembly 206 includes a tissue grasping portion 218 having first and second pluralities of prongs 220A, 220B oriented such that distal portions 222A, 222B of each of the first and second pluralities prongs can engage target tissue 224. The distal portions 222A of the first plurality of prongs 220A are engageable with a first target tissue portion 224A on a first side of a tissue opening 226, while the distal portions 222B of the second plurality of prongs 220B are engageable with a second target tissue portion 224B on a second side of the tissue opening 226.

In use, the tissue acquisition assembly 206 may be disposed adjacent to the tissue opening 226 such that the first plurality of prongs 220A are disposed on the first side of the tissue opening and the second plurality of prongs 220B are disposed on the second side of the tissue opening. In this position, the tissue grasping portion 218 can be oriented generally parallel to the tissue opening (see FIG. 2B). In addition, the tissue grasping portion 218 can be in a first, open, configuration such that the first and second pluralities of prongs 220A, 220B can be placed into engagement with the first and second target tissue portions 224A, 224B. The tissue acquisition assembly 206 can then be actuated using one or both of the first or second actuator 110, 112 (FIG. 1) to move the first and second pluralities of prongs 220A, 220B toward each other, thereby pressing the first and second target tissue portions 224A, 224B together (FIGS. 2C-2D).

The tissue acquisition assembly 206 can be actuated in any of a variety of ways. For example, the tissue acquisition assembly 206 may be spring actuated. In such a case, a spring may be positioned at or near the tip of the device 100 and may be held in compression prior to actuation. A wire can be used to release the spring, thereby closing the tissue acquisition assembly 206 to engage the target tissue portions 224A, 224B. In such embodiments, the tissue acquisition assembly 206 can be held open over the target tissue portions 224A, 224B, and may be biased closed using the spring. Retracting the wire may recompress the spring, readying the spring and the tissue acquisition assembly 206 for additional tissue engagement evolutions. In some embodiments, each of the first and second pluralities of prongs 220A, 220B may be independently actuatable by individual controls. Alternatively, the tissue acquisition assembly 206 may be actuated by a rotating shaft that rotates teeth on one side (e.g., that associated with the first plurality of prongs 220A) while an opposite side (e.g., that associated with the second plurality of prongs 220B) is held stationary. In one example, the second plurality of prongs 220B may be engaged with target tissue portion 224B and held in place, while the first plurality of prongs 220A, which are engaged with target tissue portion 220A are drawn thereto. It will be appreciated that one or both of the tissue acquisition assembly 206 and the tissue closure assembly 208 may be retracted proximally into a head portion of the endoscope 200 once a procedure is completed.

In some embodiments the first and second pluralities of prongs 220A, 220B can be offset relative to each other along a length of the tissue grasping portion 218. As can be seen in FIG. 2D, the individual prongs of the first plurality of prongs 220A are positioned so they do not directly oppose individual prongs of the second plurality of prongs 220B. Thus, when the tissue acquisition assembly 206 is moved from the first configuration to the second configuration, the distal portions 222A, 222B of the first and second pluralities of prongs 220A, 220B press sections of tissue into the spaces between opposing adjacent prongs, causing the first and second target tissue portions 224A, 224B (now pressed together) to form a wavy overlapping seam 228 (FIG. 2D) therebetween.

Once the first and second target tissue portions 224A, 224B have been pressed together to form the wavy overlapping seam 228, the tissue closure assembly 208 can be actuated to secure the tissue portions together. In one embodiment the tissue closure assembly 208 includes a suturing assembly 230 that can include a needle 232, a T-tag 234 and a suture 236 suitable for suturing the first and second target tissue portions 224A, 224B. Some or all of the tissue closure assembly 208 may be disposed within the second lumen 216 until the tissue acquisition assembly 206 has clamped the first and second target tissue portions 224A, 224B. To suture the first and second target tissue portions 224A, 224B together, a user may actuate the first or second actuator 110, 112 (FIG. 1) so that the second control element 214 causes the tissue closure assembly 208 to be moved out of the second lumen 216 to engage the first and second target tissue portions 224A, 224B. As can best be seen in FIG. 2E, the needle 232, T-tag 234 and suture 236 may be passed through alternating flaps of tissue disposed on either side of the wavy overlapping seam 228. The wavy overlapping seam 228 enables the needle 232 to pass the T-tag 234 and suture 236 through alternating the first and second target tissue portions 224A, 224B while traversing a straight line. When the needle 232 and T-tag 234 pass through a distal end 228A of the wavy overlapping seam 228, the needle can be retracted and removed via the second lumen 216, leaving the T-tag and suture 236 in place. A cinch may be deployed using known techniques to secure the suture 236 proximal end 228B of the wavy overlapping seam 228.

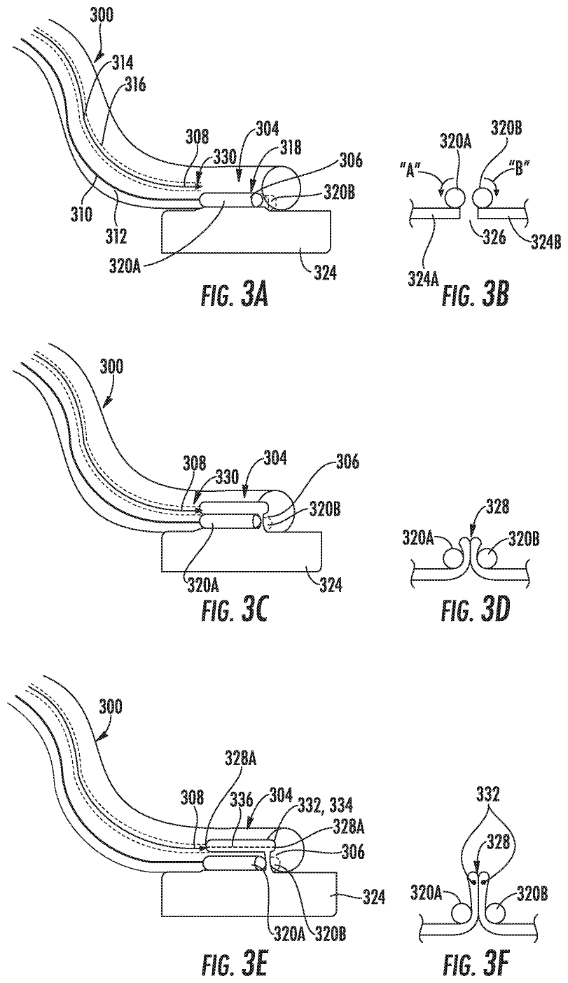

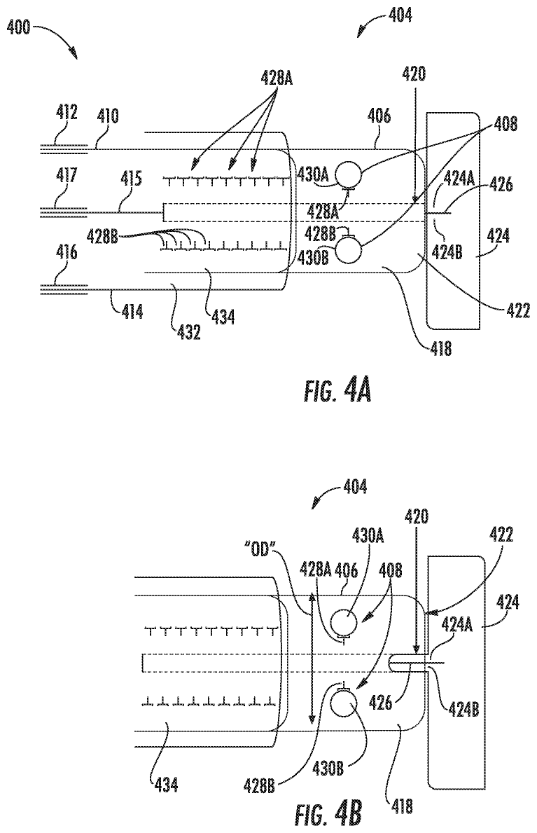

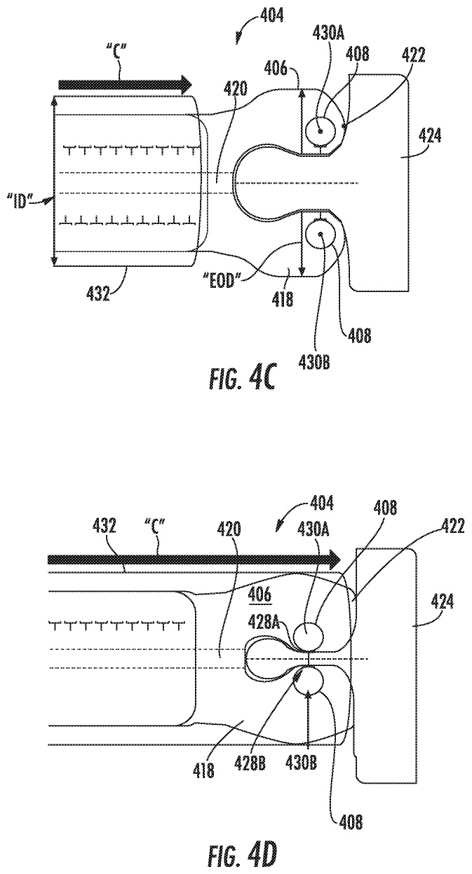

FIGS. 3A-3C illustrate an example tissue engaging portion 304 according to the present disclosure. As will be appreciated the tissue engaging portion 304 of this embodiment may be part of an endoscopic system that includes some or all the features and functionalities described in relation to the device 100 of FIG. 1. Moreover, the functionality of grasping target tissue and closing an opening in the target tissue may be conducted by a user manipulating one or both of the first and second actuators 110, 112 and/or by actuating other control mechanisms not illustrated.

The tissue engaging portion 304 may include a tissue acquisition assembly 306 and a tissue closure assembly 308. The tissue acquisition assembly 306 may be controlled via a first control element 310 disposed within a first lumen 312 in the endoscope 300, while the tissue closure assembly 308 may be controlled via a second control element 314 disposed within a second lumen 316 in the endoscope 300. The first and second control elements 310, 314 may be coupled to one or both of the first and second actuators 110, 112 (FIG. 1) to enable a user to manipulate the tissue acquisition assembly 306 and the tissue closure assembly 308 via the handle portion 12. The first and second control elements 310, 314 may comprise wires, cables, or similar structures having axial and rotational stiffness to enable the user to manipulate the tissue acquisition assembly 306 and the tissue closure assembly 308.

In the illustrated embodiment, the tissue acquisition assembly 306 includes a tissue grasping portion 318 having first and second rollers 320A, 320B for engaging target tissue 324. The first roller 320A engageable with a first target tissue portion 324A on a first side of a tissue opening 326, while the second roller 320A is engageable with a second target tissue portion 324B on a second side of the tissue opening.

In use, the tissue acquisition assembly 306 may be disposed adjacent to the tissue opening 326 such that the first roller 320A is disposed on the first side of the tissue opening and the second roller 320B is disposed on the second side of the tissue opening. In this position, the tissue grasping portion 318 can be oriented generally parallel to the tissue opening. The tissue acquisition assembly 306 can then be actuated using the first or second actuator 110, 112 (FIG. 1) to rotate the first and second rollers 320A, 320B with respect to each other. In one embodiment the first and second rollers 320A, 320B are rotated in opposite directions. For example, the first roller 320A may be rotated counterclockwise (arrow “A”), while the second roller 320B may be rotated clockwise (arrow “B”). Friction between the first and second rollers 320A, 320B and respective first and second target tissue portions 324A, 324B may cause the first and second target tissue portions to be drawn between the first and second rollers (see FIG. 3C-3D), so that the first and second target tissue portions are pressed together. In some embodiments, one or both of the first and second rollers 320A, 320B may have a roughened (e.g., sandblasted), spiked or slightly adhesive surface that enables the first and second target tissue portions 324A, 324B to be drawn between the first and second roller. In some embodiments, a friction enhancing coating may be provided on the surface of one or both of the first and second rollers 32A, 320B. When the first and second target tissue portions 324A, 324B are pressed together in the manner shown in FIGS. 3C and 3D, the tissue closure assembly 308 can be actuated to secure the tissue portions together.

In one embodiment the tissue closure assembly 308 includes a suturing assembly 330 that can include a needle 332, a T-tag 334 and a suture 336 suitable for suturing the first and second target tissue portions 324A, 324B. Some or all of the tissue closure assembly 308 may be disposed within the second lumen 316 until the tissue acquisition assembly 306 has engaged and/or clamped the first and second target tissue portions 324A, 324B. To suture the first and second target tissue portions 324A, 324B together, a user may actuate the first or second actuator 110, 112 (FIG. 1) so that the second control element 314 causes the tissue closure assembly 308 to be moved out of the second lumen 316 to engage the first and second target tissue portions 324A, 324B.

As can best be seen in FIG. 3E, the needle 332, T-tag 334 and suture 336 may be passed through the first and second target tissue portions 324A, 324B. In one non-limiting example embodiment, the needle 332 may have a corkscrew shape or other appropriate shape to enable it to engage the first and second target tissue portions 324A, 324B while traversing a path along a length of the first and second rollers 330A, 330B. When the needle 332 and T-tag 334 pass through a distal end 328A of the seam 328, the needle 332 can be retracted and removed via the second lumen 316, leaving the T-tag and suture 336 in place. A cinch may be deployed using known techniques to secure the suture 336 proximal end 328B of the seam 328.

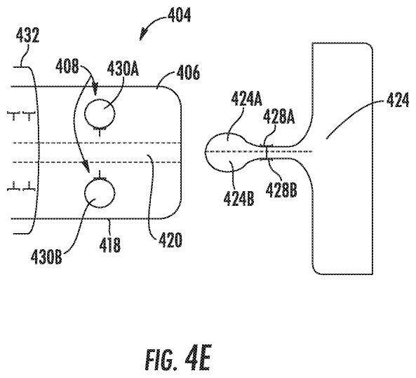

FIGS. 4A-4E illustrate an example tissue engaging portion 404 according to the present disclosure. As will be appreciated the tissue engaging portion 404 of this embodiment may be part of an endoscopic system that includes some or all the features and functionalities described in relation to the device 100 of FIG. 1. Moreover, the functionality of grasping target tissue and closing an opening in the target tissue may be conducted by a user manipulating one or both of the first and second actuators 110, 112 and/or by actuating other control mechanisms not illustrated.

The tissue engaging portion 404 may include a tissue acquisition assembly 406 and a tissue closure assembly 408. The tissue acquisition assembly 406 may be controlled via a first control element 410 disposed within a first lumen 412 in an endoscope 400, while the tissue closure assembly 408 may be controlled via a second control element 414 disposed within a second lumen 416 in the endoscope 400 (and may be coupled to a sleeve 432, the operation of which will be described in greater detail below). In the present embodiment, a third control element 415 may be disposed within a third lumen 417 and may be provided for controlling aspects of the tissue acquisition assembly 406, as will be discussed in greater detail later. The first and second control elements 410, 414 may be coupled to the first and second actuators 110, 112 (FIG. 1), while the third control element can be coupled to the first or second actuator or a third actuator or other control device to enable a user to manipulate the tissue acquisition assembly 406 and the tissue closure assembly 408 via the handle portion 12.

The first and second control elements 410, 414 may comprise wires, cables, or similar structures having axial and rotational stiffness to enable the user to manipulate the tissue acquisition assembly 406 and the tissue closure assembly 408. The third control element 415 may comprise a suction source coupled to a suction pump (not shown) associated with the handle portion 12.

In the illustrated embodiment, the tissue acquisition assembly 406 includes a distal tip portion 418 having a suction channel 420 disposed therethrough. The suction channel 420 is in fluid communication with the third control element 415 so that suction can be applied from the suction source near the handle portion 12. In one non-limiting embodiment, the suction channel 420 may be the working channel of the endoscope 400. The distal tip portion 418 may be made from a flexible material that can be radially expansible, as will be described in greater detail below, and may have a distal end 422 that can be generally flat for pressing against target tissue 424.

In use, the tissue acquisition assembly 406 may be disposed adjacent to the tissue opening 426 such that the distal tip portion 418 engages the target tissue 424 and the suction channel 420 overlies the tissue opening to engage first and second target tissue portions 424A, 424B on opposite sides of the tissue opening 426. In this position, the suction channel 420 can be oriented generally perpendicular to the tissue opening 426. The tissue acquisition assembly 406 can then be actuated using the third control element 415 to apply suction to the suction channel 420. As shown in FIG. 4B, the application of suction can cause the first and second target tissue portions 424A, 424B on opposite sides of the tissue opening 426 to be drawn into the suction channel 420. Continued application of suction via the suction channel 420 may draw additional amounts of the first and second target tissue portions 424A, 424B into the suction channel. Because the distal tip portion 418 of the tissue acquisition assembly 406 is flexible, the additional tissue drawn with the suction channel 420 may cause the distal tip portion 418 to transition from a generally cylindrical shape (see FIGS. 4A-4B), to a bulbous shape having an expanded outer diameter “EOD” that is greater than the outer diameter “OD” of the distal tip portion 418 prior to the application of suction.

When the first and second target tissue portions 424A, 424B are pressed together in the manner shown in FIG. 4C, the tissue closure assembly 408 can be actuated to secure the tissue portions together. In one embodiment the tissue closure assembly 408 includes opposing first and second staple assemblies 428A, 428B and opposing first and second hammer assemblies 430A, 430B. The first and second hammer assemblies 430A, 430B can be positioned on opposite sides of the suction channel 420, directly adjacent to respective ones of the first and second staple assemblies 428A, 428B.

To couple the first and second target tissue portions 424A, 424B together, a user may actuate the first or second actuator 110, 112 (FIG. 1) so that the second control element 414 causes a sleeve 432, which is disposed about the 406, to move toward the distal tip portion 418 of the tissue acquisition assembly 406 (i.e., in the direction of arrow “C”). The sleeve 432 may be rigid and may have an inner diameter “ID” that is larger than the “OD” of the distal tip portion 418 prior to the application of suction but is smaller than the expanded outer diameter “EOD” of the distal tip portion. As the sleeve 432 engages the distal tip portion 418, it forces the expanded distal tip portion 418 to compress (see FIG. 4D). This, in turn, causes the first and second hammer assemblies 430A, 430B to engage the first and second staple assemblies 428A, 428B and move them toward each other until they penetrate the first and second target tissue portions 424A, 424B and lock to each other, holding the first and second target tissue portions together. The second control element 414 may then be operated to retract the sleeve 432 from engagement with the distal tip portion 418, and the third control element 415 may be operated to reduce or eliminate suction applied to the suction channel 420. The distal tip portion 418 may then be retracted from the first and second target tissue portions 424A, 424B.

In some embodiments, the first and second staple assemblies 428A, 428B may have corresponding male/female conical surfaces that can mate and lock when the two are pressed together using the first and second hammer assemblies 430A, 430B. In other embodiments the first and second staple assemblies 428A, 428B are attached to each other or bent over when pressed together, fixing the first and second staple assemblies (and associated tissue portions) together.

The tissue engaging portion 404 may include a central portion 434 that houses a plurality of first and second staple assemblies 428A, 428B. In some embodiments this arrangement can allow for serial stapling of target tissue, which may be useful when closing openings in target tissue that would benefit from the application of multiple fastening points.

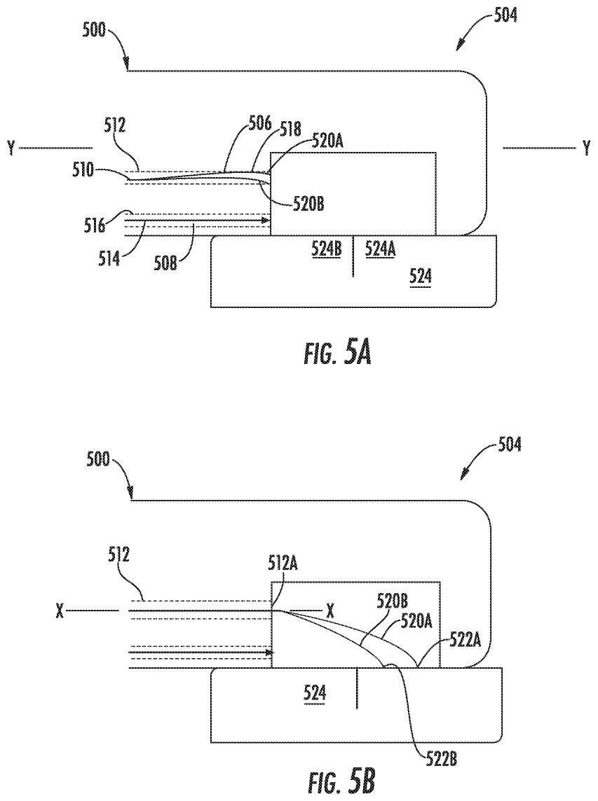

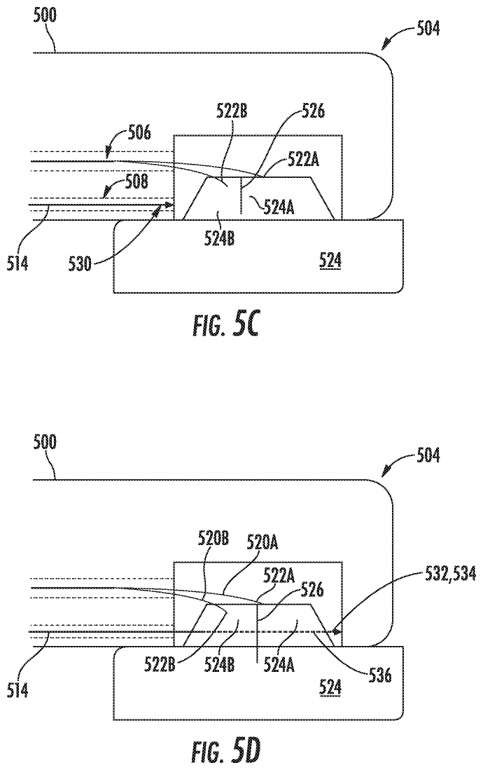

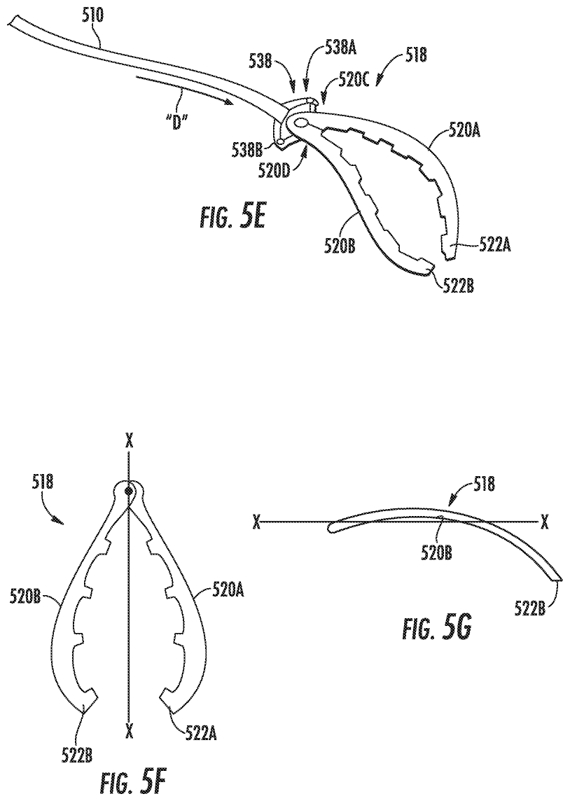

FIGS. 5A-5G illustrate an example tissue engaging portion 504 according to the present disclosure. As will be appreciated the tissue engaging portion 504 of this embodiment may be part of an endoscopic system that includes some or all the features and functionalities described in relation to the device 100 of FIG. 1. Moreover, the functionality of grasping target tissue and closing an opening in the target tissue may be conducted by a user manipulating one or both of the first and second actuators 110, 112 and/or by actuating other control mechanisms not illustrated.

The tissue engaging portion 504 may include a tissue acquisition assembly 506 and a tissue closure assembly 508. The tissue acquisition assembly 506 may be controlled via a first control element 510 disposed within a first lumen 512 in the endoscope 500, while the tissue closure assembly 508 may be controlled via a second control element 514 disposed within a second lumen 516 in the endoscope 500. The first and second control elements 510, 514 may be coupled to the first and second actuators 110, 112 (FIG. 1) to enable a user to manipulate the tissue acquisition assembly 506 and the tissue closure assembly 508 via the handle portion 12. The first and second control elements 510, 514 may comprise wires, cables, or similar structures having axial and rotational stiffness to enable the user to manipulate the tissue acquisition assembly 506 and the tissue closure assembly 508.

In the illustrated embodiment, the tissue acquisition assembly 506 includes a tissue grasping portion 518 having first and second arms 520A, 520B, such as illustrated in FIGS. 5E-5G. In some embodiments, the first and second arms 520A, 520B are oriented such that distal portions 522A, 522B of each of the first and second arms can engage target tissue 524. In some embodiments, the distal portions 522A, 522B of the first and second arms 520A, 520B, may engage a first target tissue portion 524A adjacent to a tissue opening 526, and may be used to pull that first target tissue portion toward a second target tissue portion 524B on an opposite side of the tissue opening. The tissue closure assembly 508 may then be used to close the tissue opening 526. In other embodiments, the distal portion 522A of the first arm 520A is engageable with a first target tissue portion 524A adjacent to the tissue opening 526, while the distal portion 522B of the second arm 520B is engageable with a second target tissue portion 524B adjacent to the tissue opening. The distal portions 522A, 522B can then be used to draw the first and second target tissue portions 524A, 524B together so that the tissue closure assembly 508 can close the tissue opening 526.

In some embodiments, at least a portion of the tissue grasping portion 518 may be made from a super-elastic material such as Nitinol. This can enable the tissue grasping portion 518 to be held in a relatively straight configuration when the tissue grasping portion is disposed within the first lumen 512 (see FIG. 5A). Such an arrangement can facilitate advancement of the tissue grasping portion 518 through the first lumen. When the tissue grasping portion 518 is moved out of the distal end 512A of the first lumen 512, the first and second arms 520A, 520B can re-assume their original curved shapes, which may naturally dispose the first and second arms at locations adjacent to a tissue opening 526. For example, the first and second arms 520A, 520B each has a compound curvature that positions the distal portions 522A, 522B of the first and second arms directly adjacent to, and/or in contact with, the first and second target tissue portions 524A, 524B adjacent to the tissue opening 526.

The compound curvature of the first and second arms 520A, 520B can best be seen in FIGS. 5E-5G. FIG. 5F is a plan view of the first and second arms 520A, 520B illustrating a first curvature of the first and second arms that can serve to position the first and second distal portions 522A, 522B on opposite sides of the tissue opening 526. In one embodiment this first curvature disposes the first and second arms 520A, 520B on opposite sides of the longitudinal axis X-X of the first lumen 512. FIG. 5G is a side view of the first and second arms 520A, 520B illustrating a second curvature of the first and second arms that can serve to engage the distal portions 522A, 522B of the first and second arms with the target tissue 524. In one embodiment this second curvature disposes the first and second distal portions 522A, 522B away from the longitudinal axis X-X of the first lumen 512, toward the target tissue 524.

In use, the tissue acquisition assembly 506 may be disposed adjacent to the tissue opening 526 such that the tissue opening 526 is perpendicular to the longitudinal axis Y-Y of the endoscope 500 (FIG. 5A). The tissue grasping portion 518 may be disposed in an elongated configuration within the first lumen 512. The user may actuate the first actuator 110 (FIG. 1) to move the tissue grasping portion 518 out of the distal end 512A of the first lumen 512. As the tissue grasping portion 518 exits the first lumen 512 the tissue grasping portion automatically reverts to reassume the geometry illustrated in FIGS. 5E-5G, thus positioning the first and second distal portions 522A, 522B into engagement with the target tissue 524 (FIG. 5B). In this position the tissue grasping portion 518 can be in a first, open, configuration in which the first and second arms 520A, 520B engage the first and second target tissue portions 524A, 524B. The tissue acquisition assembly 506 can be actuated using the first actuator 110 (FIG. 1) to move the first and second arms 520A, 520B toward each other, thereby pressing the first and second target tissue portions 524A, 524B together (FIG. 5C). In one non-limiting example embodiment, best illustrated in FIG. 5E, the first or second actuator 110, 112 can be used to move the first control element 510 in a first direction (arrow “D”) to cause the first and second arms 520A, 520B to engage the target tissue 524. Proximal ends 520C, 520D of the first and second arms 520A, 520B may be pivotably coupled together, and a scissor-arm mechanism 538 may couple between the first control element 510 and the first and second arms 520A, 520B. The scissor-arm mechanism 538 may include a first scissor coupling 538A connecting the first control element 510 to the first arm 520A, and a second scissor coupling 538B connecting the first control element 510 to the second arm 520B. Thus arranged, when the first control element 510 is pushed in the direction of arrow “D” the first and second arms 520A, 520B are pivoted together (e.g., to clamp tissue). By contrast, when the first control element 510 is pulled in a direction opposite that of arrow “D” the first and second arms 520A, 520B are pivoted away from each other (e.g., to release tissue).

Once the first and second target tissue portions 524A, 524B have been pressed together, the tissue closure assembly 508 can be actuated to secure the tissue portions together. In one embodiment the tissue closure assembly 508 includes a suturing assembly 530 that can include a needle 532, a T-tag 534 and a suture 536 suitable for suturing the first and second target tissue portions 524A, 524B. Some or all of the tissue closure assembly 508 may be disposed within the second lumen 516 until the tissue acquisition assembly 506 has clamped the first and second target tissue portions 524A, 524B. To suture the first and second target tissue portions 524A, 524B together, a user may actuate the first or second actuator 110, 112 (FIG. 1) so that the second control element 514 causes the tissue closure assembly 508 to be moved out of the second lumen 516 to engage the first and second target tissue portions 524A, 524B. When the needle 532 and T-tag 534 pass through the first and second target tissue portions 524A, 524B, the needle can be retracted and removed via the second lumen 516, leaving the T-tag and suture 536 in place. A cinch may be deployed using known techniques to secure the suture 536 to the target tissue 524.

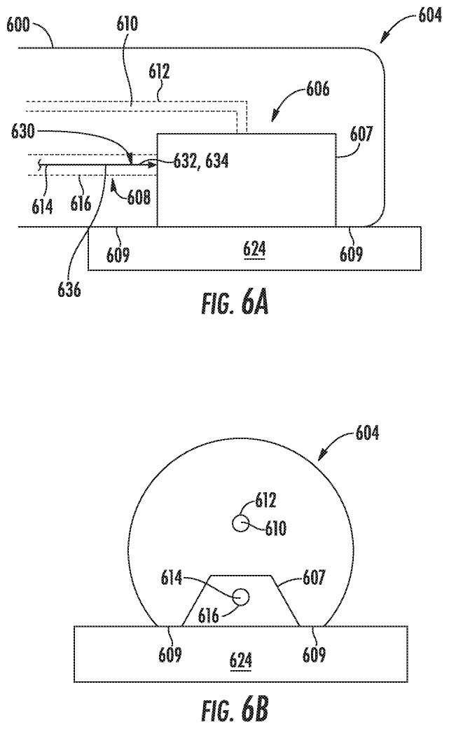

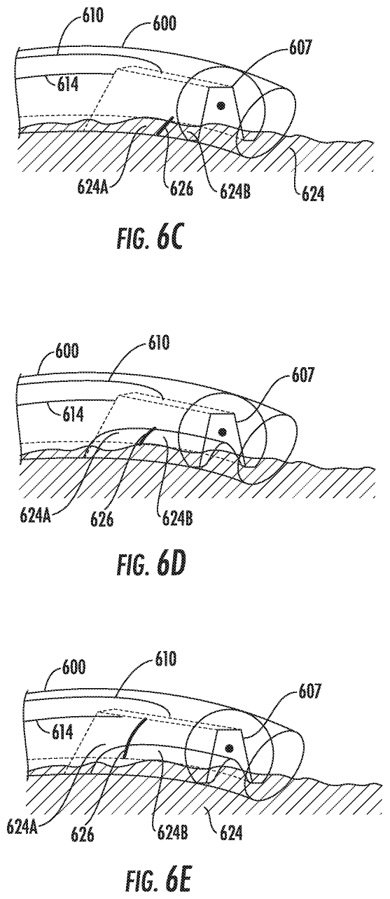

FIGS. 6A-6E illustrate an example tissue engaging portion 604 according to the present disclosure. As will be appreciated the tissue engaging portion 604 of this embodiment may be part of an endoscopic system that includes some or all the features and functionalities described in relation to the device 100 of FIG. 1. Moreover, the functionality of grasping target tissue and closing an opening in the target tissue may be conducted by a user manipulating one or both of the first and second actuators 110, 112 and/or by actuating other control mechanisms not illustrated.

The tissue engaging portion 604 may include a tissue acquisition assembly 606 and a tissue closure assembly 608. The tissue acquisition assembly 606 may be controlled via a first control element 610 disposed within a first lumen 612 in an endoscope 600, while the tissue closure assembly 608 may be controlled via a second control element 614 disposed within a second lumen 616 in the endoscope 600. The first and second control elements 610, 614 may be coupled to the first and second actuators 110, 112 (FIG. 1) to enable a user to manipulate the tissue acquisition assembly 606 and the tissue closure assembly 608 via the handle portion 12.

The first control element 610 may comprise a suction channel coupled to a suction pump (not shown) associated with the handle portion 12. The first control element 610 is illustrated as being separate from the first lumen 612, but it will be appreciated that the two could comprise the same element. The second control element 614 may comprise one or more wires, cables, or similar structures having axial and rotational stiffness to enable the user to manipulate the tissue closure assembly 608.

In the illustrated embodiment, the tissue acquisition assembly 606 includes a chamber 607, the perimeter of which may comprise surfaces 609 arranged to stabilize the tissue acquisition assembly against the target tissue so that the target tissue can be drawn up into the chamber when suction is applied thereto. The tissue acquisition assembly first control element 610 (suction channel) coupled thereto so that suction can be applied to the chamber 607 from the suction source near the handle portion 12. The first control element 610 is illustrated as connecting to the chamber 607 at a single location. It will be understood that the first control element 610 could be coupled to the chamber 607 at a plurality of locations to provide an even application of suction throughout the chamber.

In use, the tissue acquisition assembly 606 may be disposed so that the chamber 607 overlies a target tissue opening 626 (FIG. 6C) and the surfaces 609 engage the target tissue surrounding the opening. In the illustrated embodiment the chamber 607 may overlie first and second target tissue portions 624A, 624B on opposite sides of the tissue opening 626. The tissue acquisition assembly 606 can then be actuated using the first control element 610 to apply suction to the chamber 607. As shown in FIG. 6D, the application of suction can cause the first and second target tissue portions 624A, 624B on opposite sides of the tissue opening 626 to be drawn into the chamber 607. Continued application of suction via the first control element 610 may draw additional amounts of the first and second target tissue portions 624A, 624B into the chamber 607 until the first and second target tissue portions are engageable by the tissue closure assembly 608.

Once the first and second target tissue portions 624A, 624B have been drawn into the chamber 607, the tissue closure assembly 608 can be actuated to secure the tissue portions together. In one embodiment the tissue closure assembly 608 includes a suturing assembly 630 that can include a needle 632, a T-tag 634 and a suture 636 (FIG. 6A) suitable for suturing the first and second target tissue portions 624A, 624B. Some or all of the tissue closure assembly 608 may be disposed within the second lumen 616 until the tissue acquisition assembly 606 has drawn the first and second target tissue portions 624A, 624B into the chamber 607. To suture the first and second target tissue portions 624A, 624B together, a user may actuate the first or second actuator 112 (FIG. 1) so that the second control element 614 causes the tissue closure assembly 608 to be moved out of the second lumen 616 to engage the first and second target tissue portions 624A, 624B. When the needle 632 and T-tag 634 pass through the first and second target tissue portions 624A, 624B, the needle can be retracted and removed via the second lumen 616, leaving the T-tag and suture 636 in place. A cinch may be deployed using known techniques to secure the suture 636 to the target tissue 624.

Devices according to the embodiments described, and in accordance with other embodiments of the present disclosure, alone or in a system or kit or as part of a method or procedure, including with other accessories, may be used in cavities, lumens, tracts, vessels, and organs of the body, etc.

Variations, modifications, and other implementations of the present disclosure in addition to the various embodiments described herein will occur to those of ordinary skill in the art. Accordingly, the present disclosure is to be defined not by the preceding illustrative description but instead by the following claims:

What is claimed is:

1. A tissue closure system, comprising:
an endoscope having first and second lumens therethrough;
first and second control elements disposed within the first and second lumens, respectively; and
a tissue engaging portion integral with a distal end of the endoscope, the tissue engaging portion comprising a tissue acquisition assembly and a tissue closure assembly, the tissue acquisition assembly controllable via the first control element, the tissue closure assembly controllable via the second control element;
wherein the tissue engaging portion comprises a tissue grasping portion for engaging first and second target tissue portions disposed adjacent an opening in target tissue;
wherein the tissue closure assembly is configured to pierce the target tissue, and to hold the first and second target tissue portions together to close the opening in the target tissue.

2. The tissue closure system of claim 1, wherein the tissue grasping portion comprises first and second pluralities of prongs oriented such that distal portions of each of the first and second pluralities of prongs are engageable with the first and second target tissue portions;
wherein the first and second pluralities of prongs are offset relative to each other along a length of the tissue grasping portion so that when the tissue grasping portion is moved from a first configuration to a second configuration, the distal portions of the first and second pluralities of prongs are operable to press sections of tissue into spaces between opposing adjacent prongs, causing the first and second target tissue portions to form a wavy overlapping seam therebetween.

3. The tissue closure system of claim 1, wherein the tissue grasping portion comprises first and second rollers, the first roller engageable with the first target tissue portion, the second roller engageable with the second target tissue portion, the first and second rollers being rotatable in opposite directions to cause the first and second target tissue portions to be drawn between the first and second rollers to press the first and second target tissue portions together.

4. The tissue closure system of claim 3, wherein the tissue closure assembly comprises a needle comprises a corkscrew shape so that as the needle is passed along a seam between the first and second target tissue portions the needle and a fastener pass alternately through the first and second target tissue portions to close the opening.

5. The tissue closure system of claim 1, wherein the tissue grasping portion comprises a distal tip portion having a suction channel disposed therethrough, the suction channel in fluid communication with a suction source, the distal tip portion made from a flexible material for pressing against the target tissue,
wherein the distal tip portion is engageable with the target tissue such that the suction channel overlies the tissue opening to engage the first and second target tissue portions,
wherein suction applied via the suction channel causes the first and second target tissue portions to be drawn into the suction channel to press the first and second target tissue portions together.

6. The tissue closure system of claim 5, wherein the first and second target tissue portions drawn into the suction channel cause the distal tip portion to transition from a generally cylindrical shape to a shape having an expanded outer diameter “EOD” that is greater than an outer diameter “OD” of the distal tip portion prior to suction being applied via the suction channel;
the tissue grasping portion further comprising a sleeve that is movable over the distal tip portion to compress the distal tip portion, thereby causing first and second fasteners disposed in the distal tip portion to pierce the first and second tissue portions and to lock to each other, closing the opening.

7. The tissue closure system of claim 1, wherein the tissue grasping portion comprises first and second arms having first and second distal portions engageable with the first and second target tissue portions, wherein the first and second arms each has a first curvature so that the first and second arms are disposable on opposite sides of a longitudinal axis of the first lumen, and a second curvature so that the first and second arms are disposable away from the longitudinal axis and toward the target tissue.

8. The tissue closure system of claim 7, wherein the first and second arms are made from a super-elastic material, and wherein the tissue grasping portion is configurable in a first elongated configuration when the tissue grasping portion is disposed in the first lumen, and wherein when the tissue grasping portion is moved out of the first lumen the first and second arms automatically reassume the first and second curvatures.

9. The tissue closure system of claim 1, wherein the tissue acquisition assembly comprises a chamber and the first control element comprises a suction channel to apply suction to the chamber, wherein when the chamber overlies the opening in the target tissue, the suction is configured to draw the first and second target tissue portions into the chamber, pressing the first and second target tissue portions together.

10. An endoscope, comprising:
a handle including first and second actuators;
a flexible tube having an articulable distal end; and
a tissue engaging portion integral to the distal end of the flexible tube, the tissue engaging portion comprising a tissue acquisition assembly and a tissue closure assembly;
wherein the tissue engaging portion comprises a tissue grasping portion for engaging first and second target tissue portions disposed adjacent an opening in target tissue; and
wherein the tissue closure assembly is configured to pierce the target tissue, and to hold the first and second target tissue portions together to close the opening in the target tissue.

11. The endoscope of claim 10, wherein the tissue grasping portion comprises first and second pluralities of prongs oriented such that distal portions of each of the first and second pluralities of prongs are engageable with the first and second target tissue portions;
wherein the first and second pluralities of prongs are offset relative to each other along a length of the tissue grasping portion so that when the tissue grasping portion is moved from a first configuration to a second configuration, the distal portions of the first and second pluralities of prongs are operable to press sections of tissue into spaces between opposing adjacent prongs, causing the first and second target tissue portions to form a wavy overlapping seam therebetween.

12. The endoscope of claim 10, wherein the tissue grasping portion comprises first and second rollers, the first roller engageable with the first target tissue portion, the second roller engageable with the second target tissue portion, the first and second rollers being rotatable in opposite directions to cause the first and second target tissue portions to be drawn between the first and second rollers to press the first and second target tissue portions together.

13. The endoscope of claim 12, wherein the tissue closure assembly comprises a needle comprises a corkscrew shape so that as the needle is passed along a seam between the first and second target tissue portions the needle and a fastener pass alternately through the first and second target tissue portions to close the opening.

14. The endoscope of claim 10, wherein:
the tissue grasping portion comprises a distal tip portion having a suction channel disposed therethrough, the suction channel in fluid communication with a suction source, the distal tip portion made from a flexible material for pressing against the target tissue;
the distal tip portion is engageable with the target tissue such that the suction channel overlies the tissue opening to engage the first and second target tissue portions; and
suction applied via the suction channel causes the first and second target tissue portions to be drawn into the suction channel to press the first and second target tissue portions together.

15. The endoscope of claim 14, wherein the first and second target tissue portions drawn into the suction channel cause the distal tip portion to transition from a generally cylindrical shape to a shape having an expanded outer diameter “EOD” that is greater than an outer diameter “OD” of the distal tip portion prior to application of a the suction;
the tissue grasping portion further comprising a sleeve that is movable over the distal tip portion to compress the distal tip portion, thereby causing first and second fasteners disposed in the distal tip portion to pierce the first and second target tissue portions and to lock to each other, closing the opening.

16. The endoscope of claim 10, wherein the tissue grasping portion comprises first and second arms having first and second distal portions engageable with the first and second target tissue portions, wherein the first and second arms each has a first curvature so that the first and second arms are disposable on opposite sides of a longitudinal axis of a first lumen of an endoscope, and a second curvature so that the first and second arms are disposable away from the longitudinal axis and toward the target tissue.

17. The endoscope of claim 10, wherein:
the tissue acquisition assembly comprises a chamber and a first control element;
the first control element comprises a suction channel to apply suction to the chamber; and
when the chamber overlies the opening in the target tissue, the suction is configured to draw the first and second target tissue portions into the chamber, pressing the first and second target tissue portions together.

18. A method for closing an opening in tissue, comprising:
positioning a distal end of an endoscope adjacent to an opening in tissue, the distal end including an integral tissue acquisition assembly;
engaging first and second target tissue portions on opposing sides of a tissue opening using a grasping portion of the tissue acquisition assembly, the grasping portion associated with the distal end of the endo scope;
holding the first and second target tissue portions together using the grasping portion;
piercing the first and second target tissue portions using a tissue closure assembly; and
engaging the first and second target tissue portions using a fastener to close the opening in tissue.

19. The method of claim 18, wherein engaging first and second target tissue portions comprises:
engaging the first and second target tissue portions using first and second pluralities of prongs, the first and second pluralities of prongs being offset relative to each other along a length of the tissue grasping portion; and
moving the tissue grasping portion from a first configuration to a second configuration so that distal portions of the first and second pluralities of prongs press sections of tissue into spaces between opposing adjacent prongs causing the first and second target tissue portions to form a wavy overlapping seam therebetween.

20. The method of claim 18, wherein engaging first and second target tissue portions comprises applying suction to the first and second target tissue portions to draw the first and second target tissue portions into a position accessible by a tissue closure assembly of the endoscope to engage the first and second target tissue portions using the fastener.
